(19) United States
(12) Patent Application Publication
(10) Pub. No.: US 2018/0055696 A1
(43) Pub. Date: Mar. 1, 2018

(54) Nursing Pad

(71) Applicant: Crystal Smith, Bellevue, WA (US)

(72) Inventor: Crystal Smith, Bellevue, WA (US)

(21) Appl. No.: 15/681,386

(22) Filed: Aug. 20, 2017

Related U.S. Application Data
(60) Provisional application No. 62/378,665, filed on Aug. 23, 2016.

(51) Int. Cl. A61F 13/14

(52) U.S. Cl. CPC A61F 13/141; A61F 13/66

(57) ABSTRACT

This invention provides a novel solution for a nursing pad that is configured to provide direct access to an absorbent insert, resists slippage while worn inside a bra or a shirt, and effectively wick leaking breast fluid via an undergarment pad assembly comprising a receptacle with a pocket, at least one absorbent insert wherein the at least one absorbent insert is retained and semi-enveloped within the pocket of the receptacle, and may include at least one non-slip surface on a backside of the receptacle.

### CROSS-REFERENCE TO RELATED APPLICATIONS

The present application claims priority to provisional patent Application No. 62/378,665 filed Aug. 23, 2016 the contents of which are incorporated herein by reference.

### FIELD OF THE INVENTION

This invention is in the field of nursing pads particularly absorbent pads that are used to keep breast milk from leaking or seeping through a woman's bra or shirt.

### BACKGROUND INFORMATION

Women need nursing pads to keep breast milk from leaking or seeping through a bra or a shirt. Current nursing pad solutions are lacking because the pads do not enable flexibility to handle light versus heavy leakage. For example, thinner pads are suitable for light leakage, but would not be sufficient for heavy leakage. Likewise a heavy pad would be too bulky to wear during periods of light leakage. Current nursing pad solutions are also uncomfortable due to the pad slipping inside the bra or shirt which is also a cause for leakage. Current nursing pads also lack the ability to remove absorbent insert for an ecofriendly disposal and they lack the ability to properly wash, air dry and sanitize a reusable absorbent insert in a waterproof design. There is a need for a more effective, comfortable, user-friendly, and eco-friendly nursing pad.

This invention provides a novel solution for a nursing pad that is configured to provide direct access to an absorbent pad, resists slippage while worn inside a bra or a shirt, and effectively wicks leaking breast milk via a single or multi-piece retainer comprised of wicking fabric to provide a dry feeling to the breast even during periods of heavy leakage.

### BRIEF DESCRIPTION OF THE DRAWINGS

Features and advantages of the claimed subject matter will be apparent from the following detailed description of embodiments consistent therewith, which description should be considered with reference to the accompanying drawings, wherein:

FIG. 1 illustrates the nursing pad components in accordance with the teachings of the present invention;

FIG. 2 illustrates a front view of the nursing pad assembly in accordance with the teachings of the present invention;

FIG. 3 illustrates a front view of a receptacle in accordance with the teachings of the present invention;

FIG. 4 illustrates a rear view of a receptacle in accordance with the teachings of the present invention;

FIG. 5 illustrates a front and cross sectional view of the nursing pad assembly in accordance with the teachings of the present invention;

FIG. 6 illustrates a rear view of a receptacle including an optional decorative lace fabric in accordance with the teachings of the present invention;

FIG. 7 illustrates an example of a method to make a nursing pad in accordance with the teachings of the present invention;

FIG. 8 illustrates an example of a method to make a nursing pad in accordance with the teachings of the present invention; and

FIG. 9 illustrates an example of a method to make a nursing pad in accordance with the teachings of the present invention;

### DETAILED DESCRIPTION OF THE INVENTION

The following describes the details of the invention. Although the following description will proceed with reference being made to illustrative embodiments, many alternatives, modifications, and variations thereof will be apparent to those skilled in the art. Accordingly, it is intended that the claimed subject matter be viewed broadly. Examples are provided as reference and should not be construed as limiting. The term “such as” when used should be interpreted as “such as, but not limited to.”

A nursing pad 100 comprises a insert 300 wherein the insert 300 includes a first strip of material 251, a second strip of material 252, and a third strip of material 253 attached to an outer perimeter edge of a rear surface 701 to form a pocket 855 with an opening 251. At least one absorbent insert 300 is inserted into the pocket via the opening 251. The nursing pad 100 may also include a first strip of material attached to the rear surface 701 via a first seam 703, the second strip of material may be attached to the rear surface 701 via a second seam 705, and the third strip may be attached to the rear surface 701 via a third seam 707. The second strip of material may be position so that a portion of the second strip of material overlaps the first strip of material, and the third strip of material may be position so that a portion of the third strip of material overlaps the second strip of material and so that a portion of the third strip of material is tucked inside of the first strip of material. The back surface may be a circular disk of material with a diameter of about 2-10 inches and the first strip of material, the second strip of material, and the third strip of material may be formed as a chord with a diameter similar to the back surface and the size of the chords when assembled form the opening 251, and when combined with the rear surface 701 form the pocket. The pocket may be sized such that the absorbent insert 300 is partially folded and inserted into the pocket and then unfolded once inserted into the pocket and the absorbent insert 300 is retained and semi-enveloped within the insert 300 with a portion of the absorbent insert 300 exposed through the opening 251. The insert 300 may be made from a fluid resistant material. The absorbent inserts may be comprised of at least a single ply of an absorbent material. The absorbent insert 300 may comprise paper, sponge, organic fabric, synthetic fabric, super absorbent polymers, or a combination thereof. The absorbent insert 300 may also be shaped as a thin disk with a diameter ranging from one to ten inches, or as needed to fit within the insert 300 and pocket. The absorbent insert 300 may have a planar shape comprising a polygon or curved shape. The first strip of material, the second strip of material, and the third strip of material may comprise a wicking fabric material consisting of organic or synthetic fibers. The rear surface 701 may include at least one non-slip feature.

An undergarment pad assembly comprises a insert 300 formed with a first flap attached to a mid-ring 801 via a first seam 703, a second flap attached to the mid-ring 801 via a second seam 705, a third flap attached to the mid-ring 801 via a third seam 707, and a rear surface 701 attached to the mid-ring 801 forming a pocket with an opening 251 between the flaps and the rear surface 701 and at least one absorbent insert 300 inserted into the pocket via the opening 251. The second flap may be positioned so that a portion of the second flap overlaps a portion of the first flap, and the third flap is positioned so that a portion of the third flap overlaps a portion of the second flap and so that a portion of the third flap is tucked inside of a portion of the first flap. The rear surface 701 may be a circular disk with a diameter of about 4-10 inches and the first flap, the second flap, and the third flap are formed as a chord with a diameter similar to the rear surface 701 and the size of the chords when assembled form an opening 251, and when combined with the rear surface 701 form a pocket. The pocket may be sized such that the absorbent insert 300 may be partially folded and inserted into the pocket via the opening 251 and then unfolded once inserted into the pocket. The mid-ring 801 may be shaped as an annular ring with a major and minor diameter and the major diameter may be sized to be similar to the outside diameter of the rear surface 701. The thickness of the mid-ring 801 may be sized so the pocket can accommodate more than one absorbent insert 300. The mid-ring 801 may be formed from a bendable material such that the insert 300 can be bent into a contoured shape to better fit along a breast.

A nursing pad 100 comprises a front layer formed from a single layer of material, wherein the front layer 901 includes a cutout feature 903, and a rear layer 905 attached to the front layer 901 at an outer seam of the front layer 901 forming a pocket 855. The cutout feature 903 may be an opening 251 to the pocket 855, and the cutout feature 903 may be sized for inserting and removing an absorbent insert 300 into the pocket 855, and the cutout feature 903 may be sized such that an exposed portion of the absorbent insert 300 is in direct contact with a breast.

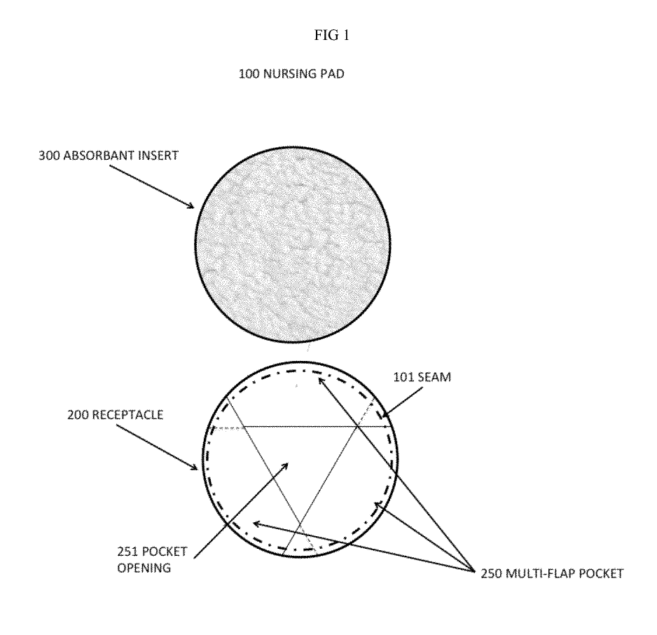

FIG. 1 illustrates a nursing pad 100. The nursing pad 100 includes a receptacle 200 and at least one absorbent insert 300. The receptacle 200 includes a soft moisture wicking front surface which provides both comfort and a stay dry feel against the skin of the user. For example, the front surface of the receptacle 200 may be made of moisture wicking micro fleece material comprised of microfiber polyester yarns. The rear portion of the receptacle 200 may be made of an inner laminated water resistant fabric and may include an optional non-slip feature 400 on the outer surface to prevent slippage during use. Furthermore, the receptacle 200 securely retains the absorbent insert 300 or inserts and allows the absorbent insert 300 to be easily removed for an eco-friendly disposal, or for washing and sanitizing of reusable inserts to promote an eco-friendly alternative to existing disposable pads. Likewise the receptacle 200 is also reusable and washable with the absorbent insert 300 removed. The nursing pad 100 is lightweight and durable enabling cleaning and drying by either machine or hand washing with a quick pat dry. The nursing pad 100 is discreet and can be worn beneath an undergarment, shirt, dress, or other wardrobe.

The receptacle 200 may be formed by combining a first portion of material and multiple flap 250 retaining pieces of material to form a pocket 855. The first portion of fabric and the multiple flap 250 retaining pieces of material may be joined by seam at the outer perimeter edge. The seam may include, for example a stitch, fabric adhesive, material fusion, or any other type of fastening feature.

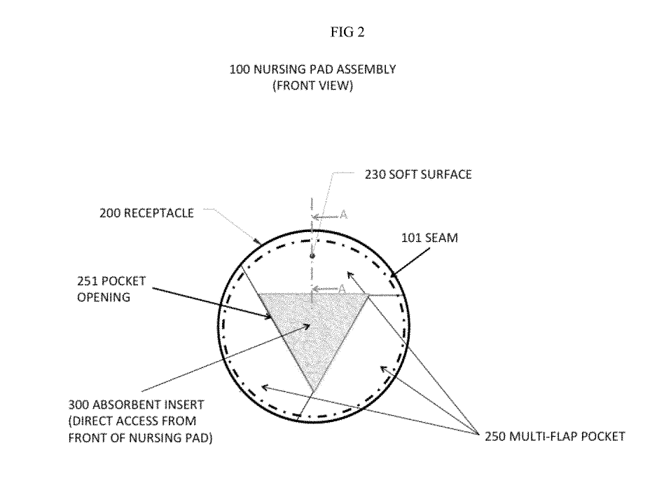

FIG. 2 illustrates a front view of a nursing pad 100 assembly. The front surface of the receptacle 200 includes a soft surface 230. The receptacle 200 may be formed from multiple flap 250 retaining pieces attached to a rear surface 701 to form a pocket 855. The pocket 855 may be sized to hold the absorbent insert 300. The multiple flap 250 retainers may be assembled to form an opening 251 in the pocket 855. For example, FIG. 2 illustrates a nursing pad 100 within a circular shaped receptacle 200 that includes three flaps 250. Each flap 250 may be formed from a portion of material formed in the shape of a segment of a circle. A circular edge of the segment of each flap 250 retainer is attached to a circular edge of the receptacle 200, for example via the seam. The straight edge of the segment remains free to form the pocket 855. The size of each segment is optimized so that the diameter of the circular edge of each flap 250 retainer about matches the diameter of the circular edge of the rear portion of the receptacle 200. The size of the segment including the distance from the straight edge of the segment to the furthest tangent of the circular edge of the circle is optimized to form an opening 251. The size of the opening 251 is optimized to enable at least one absorbent insert 300 to be placed inside the receptacle 200 thus enabling direct access to the absorbent insert 300 from the front surface of the nursing pad 100. For example the shape of the opening 251 in the pocket 855 may form a triangle with a portion of the absorbent insert 300 exposed (as shown in FIG. 2).

The absorbent insert 300 can be optimized so only the portion of the absorbent insert 300 that is exposed against the breast via the pocket 855 includes a material optimized for contact with the nipple of the breast. For example the portion of the absorbent insert 300 that is exposed against the nipple may be made of a soft, hypoallergenic, sanitized, comfortable material. The opening 251 in the pocket 855 enables direct access for rapid and maximum absorption and easy access to the absorbent insert 300 for insertion, replacement, washing and sanitizing. The absorbent insert 300 can be made from materials optimized to quickly absorb and hold leaking fluids. For example, the absorbent insert 300 may be made of a composite material such 70% bamboo and 30% organic cotton. Furthermore the absorbent insert 300 may be combined with an optimum inner blend of cellulosic fibers interspersed with polyester nylon. The absorbent insert 300 may be made from a bamboo velour lining to provide a soft skin touch feel to relieve painful adherence to the nipple. The absorbent insert 300 may include various textures such as dimple & fiber technology to optimize the absorption and holding of leaking fluids when the nursing pad 100 is compressed.

In addition, the nursing pad 100 may also include more than one absorbent insert 300 layered to increase the absorbing capacity of the nursing pad 100. The nursing pad 100 assembly may also be able to accommodate different absorbent inserts 300. For example, a thinner absorbent insert 300 may be used to accommodate a lower absorbing capacity. Likewise a thicker insert may be used to accommodate a higher absorbing capacity. The material type of the absorbent insert 300 may also be changed to accommodate different absorbing capacities.

The nursing pad 100 components may be made from a fluid resistance fabric including at least one of PUL lined fabric, thermoplastic fabric composite, thermoplastic films, synthetic fibers, plastic lined fabric, nylon, bio-degradable polymer lined fabric, or combinations thereof. The rear surface 701, and flaps 250 may also be made of any other type of fluid resistant biodegradable polymer coating technology or a plant based natural polymer that is eco-friendly.

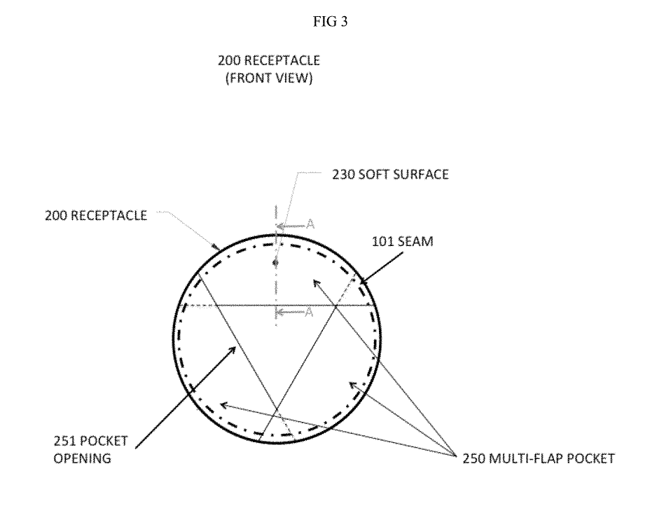

FIG. 3 illustrates the front view of a receptacle 200. The figure shows one embodiment of the invention with a receptacle 200 formed with a circular shape that is planar. The shape of the receptacle 200 may take other forms such as multi-sided polygons. In addition, the shape of the absorbent insert 300 and receptacle 200 may be non-planar. For example the shape of the nursing pad 100 may be concave to enable the nursing pad 100 to fit more comfortably against the shape of the breast. A concave shape may also enable the nursing pad 100 to capture and further prevent leaking fluids. Furthermore the diameter of the nursing pad 100 and concave depth of the nursing pad 100 can be designed for different breast sizes by varying the geometry of the components. The shape of the multi-flap 250 retainers forming the pocket 855 may also vary based on the shape of the receptacle 200 and the shape of the opening 251 desired. For example each flap 250 may be formed by a crescent shape in order to yield an opening 251 with a more circular shape, or multiple segments can be used to form other polygon shapes. The receptacle 200 may be made from a water proof, breathable, soft, flexible, and durable material such a polyurethane laminate fabric (PUL) or the like.

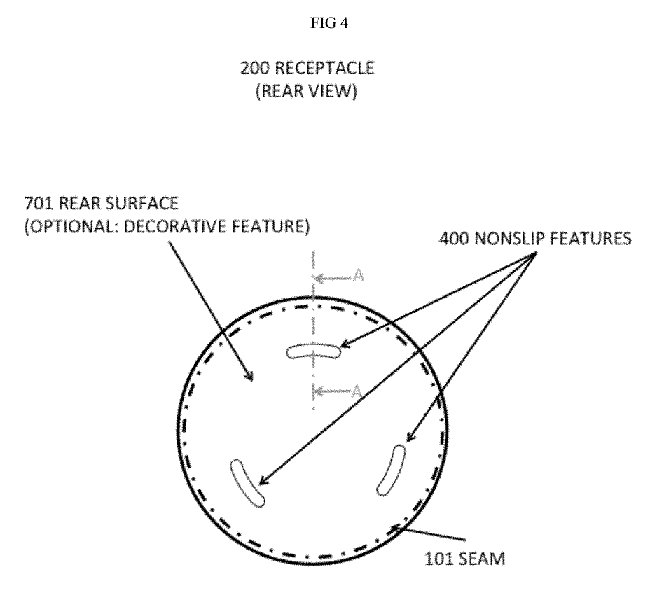

FIG. 4 illustrates a rear surface 701 of a receptacle 200. The rear surface 701 may include at least one nonslip feature. The nonslip features may be optimally placed on the back surface of the receptacle 200 such that the nursing pad 100 will not slip when worn. The nonslip feature maybe sized to be to cover a portion or the entire back surface of the receptacle 200. The nonslip features may be made of a material with a higher coefficient of friction than the backside surface of the receptacle 200. For example, the nonslip feature/s may be made from a polymer material with a textured surface and sized accordingly to produce a sufficient coefficient of friction to firmly hold the nursing pad 100 in place. The firmly held nursing pad 100 enables the absorbent material exposed via the pocket 855 to be in contact with the nipple of the breast. The non-slip feature 400 may be attached to the rear surface 701 of the receptacle 200 using a permanent fastening scheme such as but not limited to an adhesive bond or stitching. The non-slip feature 400 may also cover the entire rear surface 701. The non-slip feature 400 may also be a fabric decorative layer 450, such as the decorative lace material, attached to the rear surface 701 by an outer diameter seam. The non-slip feature 400 may be optional with a nursing pad 100 including the non-slip feature 400 and other configurations without the non-slip feature 400.

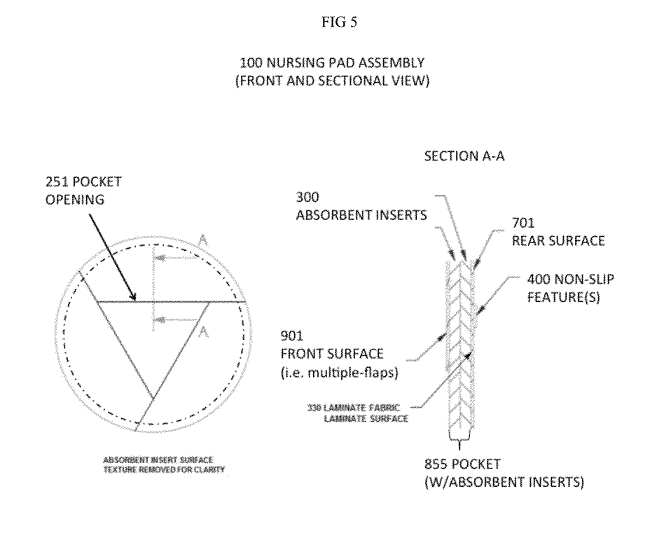

FIG. 5 illustrates a cross sectional view of a nursing pad 100 taken at the section marked A-A. The cross section shows the nursing pad 100 including the receptacle 200 with two absorbent inserts 300 placed inside of the pocket 855 of the receptacle 200. More than two absorbent inserts 300 can be used when the receptacle 200 is configured to accept more absorbent inserts 300. For example, the pocket 855 in the receptacle 200 can be configured to fit three or more absorbent inserts 300. The cross sectional view also illustrates the optional at least one non-slip feature 400 on the rear surface 701 of the receptacle 200. The outer perimeter seam holds the front rear surface 701 of the receptacle 200 along with each of the flap 250 retainers. The absorbent inserts 300 may not be fixed to the nursing pad 100 via the seam in order to allow the absorbent insert 300 from being easily removed and replaced.

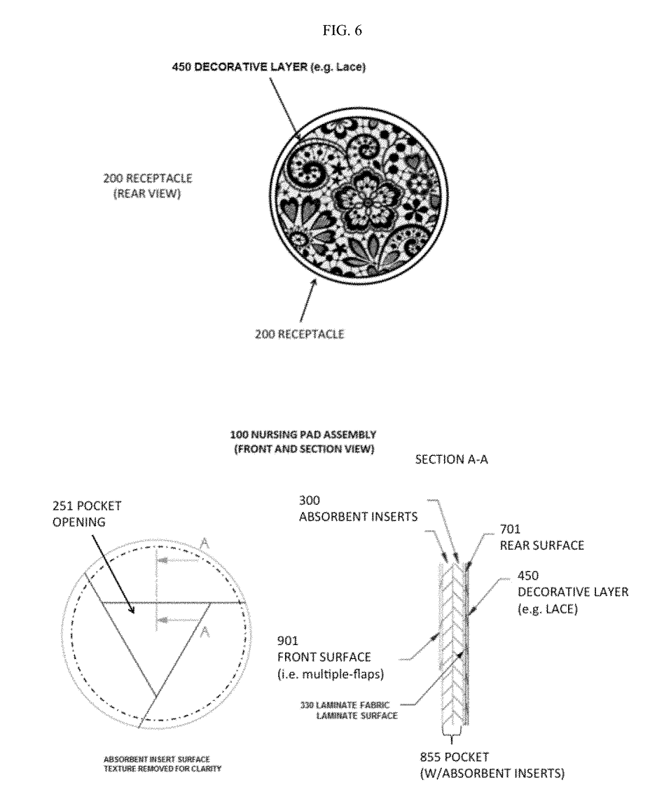

FIG. 6 is a rear view of a receptacle 200 where the rear surface 701 includes a fanciful decorative layer 450. For example the decorative layer 450 may include a lace material. The lace material may be attached to the rear surface 701 of the receptacle 200 using a permanent or temporary fastening scheme such as and adhesive, stitching, glue, or a hook-and-loop fastener or via the perimeter stitch seam. A temporary fastening scheme (e.g. a hook-and-loop fastener) may be used to allow the fanciful design to be interchangeable. For example, the fanciful design layer may be changeable to accommodate different colors, textures, and/or patterns. The fanciful design may also be printed onto the back surface of the receptacle 200. The fanciful design may also include various colors and styles to match different colors and styles of clothing thus making the nursing pad 100 less conspicuous. The decorative layer 450 may also include the non-slip properties or features described herein.

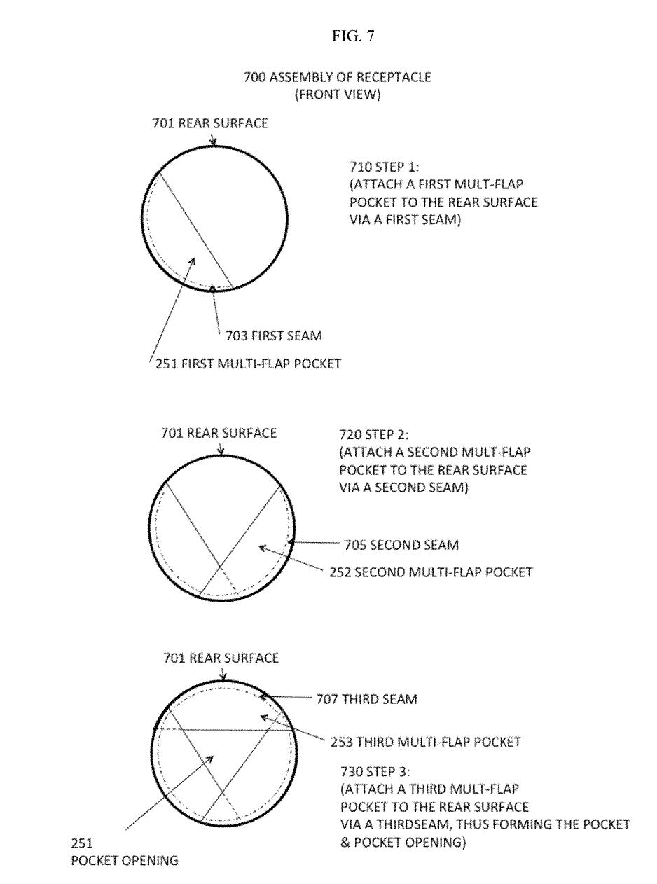

FIG. 7 includes an example of a method to assemble a receptacle 200. First a first flap retainer 251 is attached to the rear surface 701 of the receptacle 200 via a first seam 703. Next, a second flap retainer 252 is attached to the rear surface 701 of the receptacle 200 via a second seam 705. The second flap retainer 252 is positioned so that a portion of it overlaps the first flap retainer 251. Next, a third flap retainer 253 is attached to the rear surface 701 of the receptacle 200 via a third seam 707. The third flap retainer 253 is positioned so that a portion of the third flap retainer 253 overlaps the second flap retainer 252 and such that a portion of the third flap retainer 253 is tucked inside of the first flap retainer 251. The multiple flaps (e.g. 251, 252, and 253) and rear surface 701 of the receptacle 200 may be formed from the type of materials and fabrics disclosed herein. The seams (e.g. 703, 705, 707) disclosed herein may be formed using an adhesive, glue, and/or stitch. The multiple flaps (e.g. 251, 252, and 253) may be formed as a chord with a diameter similar to the back surface of the receptacle 200. The size of the chords are optimized such that when assembled the first flap retainer 251, second flap retainer 252, and third flap retainer 253 form an opening 251 and when combined with the rear surface 701 of the receptacle 200 form a pocket 855. The pocket 855 is sized such that an absorbent insert 300 may be partially folded and inserted into the pocket 855 and then unfolded once inserted into the pocket 855. Finally, the absorbent insert 300 can be inserted into the pocket 855 to form the nursing pad 100.

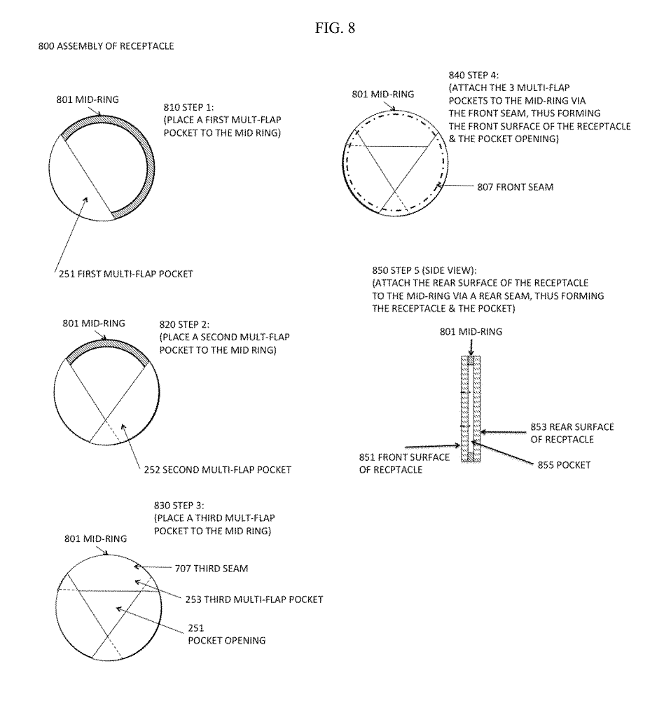

FIG. 8 includes an example of a method to assemble a receptacle 200. First, a first flap retainer 251 is attached to a mid-ring 801 via a first seam. Next, a second flap retainer 252 is attached to the mid-ring 801 via a second seam. The second flap retainer 252 is positioned so that a portion of it overlaps the first flap retainer 251. Next, a third flap retainer 253 is attached to the mid-ring 801 via a third seam 707. The third flap retainer 253 is position so that a portion of the third flap retainer 253 overlaps the second flap retainer 252 such that a portion of the third flap retainer 253 is tucked inside of the first flap retainer 251. Next, the rear surface 701 of the receptacle 200 is attached to the mid-ring 801 to form a pocket 855 between the multiple flaps (e.g 251, 252, 253) and the rear surface 701. The multiple flaps (e.g 251, 252, 253) and mid-ring 801 may be formed from the type of materials and fabrics disclosed herein. The seams disclosed herein may be formed using an adhesive, glue, and/or stitch. The multiple flaps (e.g 251, 252, 253) may be formed as a chord with a diameter similar to the rear surface 701 of the receptacle 200. The size of the chords are optimized such that when assembled the first flap 250 retainer, second flap 250 retainer, and third flap 250 retainer form an opening 251 and when combined with the rear surface 701 of the receptacle 200 form a pocket 855. The pocket 855 is sized such that an absorbent insert 300 may be partially folded and inserted into the pocket 855 and then unfolded once inserted into the pocket 855. Finally, the absorbent insert 300 can be inserted into the pocket 855 to form the nursing pad 100.

The mid-ring 801 may be shaped as an annular ring with a major and minor diameter. The major diameter is sized to be similar to the outside diameter of the back surface of the receptacle 200. The thickness of the mid-ring 801 may be sized to accommodate one or more absorbent inserts 300. The mid-ring 801 may also be formed from a pliable material such that the mid-ring 801 can be bent into a contoured shape to better fit along a breast. For example, the mid-ring 801 may include a bendable wire embedded within it that enables the mid-ring 801 to be bent into the contoured shape.

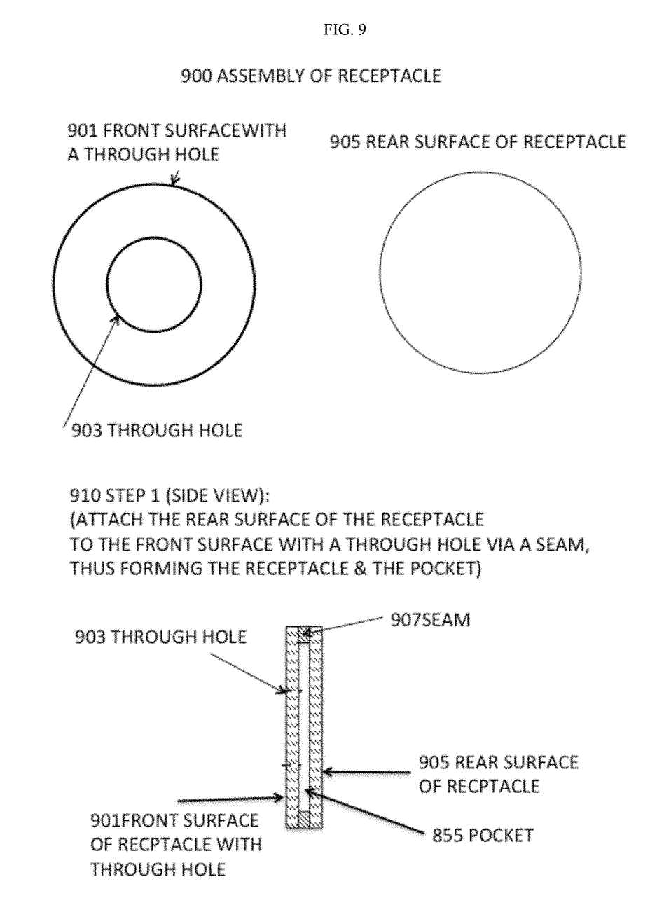

FIG. 9 includes an example of a method to assemble a receptacle 200 and a nursing pad 100. First a front surface 901 with a cutout feature 903 (e.g. a through hole) is attached to the rear surface 905 of the receptacle 200 via a first seam 907 to form a pocket 855 between the front surface 901 and the rear surface 903. The front surface 901 may include a front layer formed from a single layer of material, wherein the front layer includes a cutout feature 903. The rear surface 905 may include a rear layer attached to the front layer at an outer seam 907 of the front layer forming a pocket 855. The cutout feature 903 may be an opening to the pocket 855. The cutout feature 903 may be sized for inserting and removing an absorbent insert 300 into the pocket 855. The cutout feature 903 may be further sized such that an exposed portion of the absorbent insert 300 is in direct contact with a breast to better absorb leaking breast milk. The front surface 901 and rear surface 905 may be formed from the type of materials and fabrics disclosed herein. The seam 907 may be formed using an adhesive, glue, and/or stitch. The front surface 901 may be formed as an as an annular ring with a major and minor diameter. The major diameter may be sized to be similar to the outside diameter of the rear surface 905 of the receptacle. The minor diameter may be sized such that an absorbent insert 300 may be partially folded and inserted into the pocket 855 and then unfolded once inserted into the pocket 855. Finally, the absorbent insert 300 can be inserted into the pocket 855 to form the nursing pad 100. Additionally, the front surface 901 may be attached to a mid-ring 801, as previously disclosed herein, with the rear surface 905 also attached to the mid-ring 801 to form a pocket 855 between the front surface 901 and the rear surface 905.

The terms and expressions, which have been employed herein, are used as terms of description and not of limitation, and there is no intention, in the use of such terms and expressions, of excluding any equivalents of the features shown and described (or portions thereof), and it is recognized that various modifications are possible within the scope of the claims. Other modifications, variations, and alternatives are also possible. Accordingly, the claims are intended to cover all such equivalents.

1. A nursing pad comprising:
a receptacle wherein the receptacle comprises a first strip of material, a second strip of material, and a third strip of material attached to an outer perimeter edge of a rear surface to form a pocket with an opening; and
at least one absorbent insert inserted into the pocket via the opening.

2. The nursing pad of claim 1 wherein the first strip of material is attached to the rear surface via a first seam, the second strip of material is attached to the rear surface via a second seam, and the third strip is attached to the rear surface via a third seam.

3. The nursing pad of claim 1 wherein the second strip of material is position so that a portion of the second strip of material overlaps the first strip of material, and the third strip of material is position so that a portion of the third strip of material overlaps the second strip of material and so that a portion of the third strip of material is tucked inside of the first strip of material.

4. The nursing pad of claim 1 wherein the back surface is a circular disk of material with a diameter of about 4-10 inches and first strip of material, the second strip of material, and the third strip of material are formed as a chord with a diameter similar to the back surface and the size of the chords when assembled form the opening, and when combined with the rear surface form the pocket.

5. The nursing pad of claim 1 wherein the pocket is sized such that the absorbent insert is partially folded and inserted into the pocket and then unfolded once inserted into the pocket and the absorbent insert is retained and semi-enveloped within the receptacle with a portion of the absorbent insert exposed through the opening.

6. The nursing pad of claim 1 wherein the receptacle is made from a fluid resistant material.

7. The nursing pad of claim 1 wherein the absorbent insert is comprised of at least a single ply of an absorbent material.

8. The nursing pad of claim 1 wherein the absorbent insert comprises paper, sponge, organic fabric, synthetic fabric, super absorbent polymers, or a combination thereof.

9. The nursing pad of claim 1 wherein the absorbent insert is a disk.

10. The nursing pad of claim 1 wherein the absorbent insert has a planar shape comprising a polygon or curved shape.

11. The nursing pad of claim 1 wherein the first strip of material, the second strip of material, and the third strip of material comprise a wicking fabric material consisting of organic or synthetic fibers.

12. The nursing pad of claim 1 wherein the rear surface includes at least one non-slip feature.

13. An undergarment pad assembly comprising:
a receptacle formed with a first flap attached to a mid-ring via a first seam, a second flap attached to the mid-ring via a second seam, a third flap attached to the mid-ring via a third seam, and a rear surface attached to the mid-ring forming a pocket with an opening between the flaps and the rear surface; and
at least one absorbent insert inserted into the pocket via the opening.

14. The nursing pad of claim 13 wherein the second flap is positioned so that a portion of the second flap overlaps a portion of the first flap, and the third flap is positioned so that a portion of the third flap overlaps a portion of the second flap and so that a portion of the third flap is tucked inside of a portion of the first flap.

15. The nursing pad of claim 13 wherein the rear surface is a circular disk with a diameter of about 4-10 inches and the first flap, the second flap, and the third flap are formed as a chord with a diameter similar to the rear surface and the size of the chords when assembled form an opening, and when combined with the rear surface form a pocket.

16. The nursing pad of claim 13 wherein the pocket is sized such that the absorbent insert may be partially folded and inserted into the pocket via the opening and then unfolded once inserted into the pocket.

17. The nursing pad of claim 13 wherein the mid-ring is shaped as an annular ring with a major and minor diameter and the major diameter is sized to be similar to the outside diameter of the rear surface.

18. The nursing pad of claim 13 wherein the thickness of the mid-ring is sized so the pocket can accommodate more than one absorbent insert.

19. The nursing pad of claim 13 wherein the mid-ring is formed from a bendable material such that the receptacle can be bent into a contoured shape to better fit along a breast.

20. A nursing pad comprising:
a front layer formed from a single layer of material, wherein the front layer includes a cutout feature;
a rear layer attached to the front layer at an outer seam of the front layer forming a pocket;
wherein the cutout feature is an opening to the pocket, and the cutout feature is sized for inserting and removing an absorbent insert into the pocket, and the cutout feature is further sized such that an exposed portion of the absorbent insert is in direct contact with a breast.
